(19) United States
(12) Patent Application Publication
(10) Pub. No.: US 2026/0034254 A1
(43) Pub. Date: Feb. 5, 2026

(54) Disinfection Device and Associated Disinfection Process

(71) Applicant: GERMITEC, Bordeaux (FR)

(72) Inventors: Fanny POULON, Bures Sur Yvette (FR); Jérémy DALLARD, Talence (FR); Maxime BERTHIAS, Cadarsac (FR); Aimrane HAMDOU, Bordeaux (FR)

(21) Appl. No.: 19/283,692

(22) Filed: Jul. 29, 2025

(30) Foreign Application Priority Data
Jul. 31, 2024 (FR) FR2408458

(51) Int. Cl. A61L 2/10; A61L 2/24

(52) U.S. Cl. CPC A61L 2/10; A61L 2/24; A61L 2202/11; A61L 2202/122; A61L 2202/24

(57) ABSTRACT

The invention relates to a disinfection device (10) for a medical instrument comprising a disinfection volume (24) delimited by at least one wall (22A-F), a UV radiation generation device (26) configured to generate UV radiation in the disinfection volume (24), and an identification unit (28) positioned to read identification information of a medical instrument only when the medical instrument is positioned in the disinfection volume (24).

The present invention relates to a disinfection device for a medical instrument and an associated disinfection process.

To avoid any contamination, it is necessary to disinfect a medical instrument between two uses.

To this end, it is known from the prior art to disinfect medical instruments in disinfection devices defining a closed disinfection volume. Such devices comprise, for example, a device for generating UV (ultraviolet) radiation, allowing disinfection of medical instruments, more particularly high-level disinfection.

It is known to identify the medical instrument before placing it in the disinfection device to ensure traceability of the disinfection.

However, such identification is not entirely satisfactory, as there are risks of identification errors. In particular, there is a risk that the identified medical instrument and the one placed in the device are not the same. For example, nothing ensures that the instrument placed in the device is the same as the one previously identified.

The aim of the invention is to propose a disinfection device ensuring improved and reliable traceability of the disinfection.

To this end, the invention relates to a disinfection device for a medical instrument comprising:
a disinfection volume delimited by at least one wall,
a UV radiation generation device configured to generate UV radiation in the disinfection volume, and
an identification unit positioned to read identification information of a medical instrument only when the medical instrument is positioned in the disinfection volume.

By means of the identification unit, the medical instrument is identified only when it is positioned in the device. Thus, the identified instrument is certainly the same as the one being disinfected.

According to other advantageous aspects of the invention, the disinfection device comprises one or more of the following features, taken alone or in any technically possible combination:
The disinfection device comprises a support configured to hold the medical instrument inside the disinfection volume.
The medical instrument comprises an identification module comprising the identification information of the medical instrument, the identification unit defining an identification volume, the support being configured to hold the medical instrument so as to position the identification module attached to the medical instrument in the identification volume of the identification unit.
The identification unit of the medical instrument comprises a radio-identification reader defining the identification volume.
The disinfection device comprises a processor configured to associate the identification information of the medical instrument with disinfection characterization information.
The disinfection characterization information comprises at least the disinfection status.
The disinfection device comprises an additional identification unit for the user and/or the patient arranged outside the disinfection volume for acquiring identification information of the user of the medical instrument and/or identification of the patient for whom the medical instrument was used.
The disinfection device also comprises a display and/or printing and/or export unit for the identification information of the medical instrument.

The invention also relates to a disinfection process for a medical instrument comprising the following successive steps:
a. providing a device as described above,
b. positioning the medical instrument inside the disinfection volume,
c. reading the identification information of the medical instrument in the disinfection volume by the identification unit, and
d. starting a disinfection cycle of the medical instrument only when identification information has been read by the identification unit.

Advantageously, the process further comprises a step of associating the identification information of the medical instrument with disinfection characterization information.

The invention will become clearer upon reading the following description, given solely by way of non-limiting example, and made with reference to the drawings wherein:

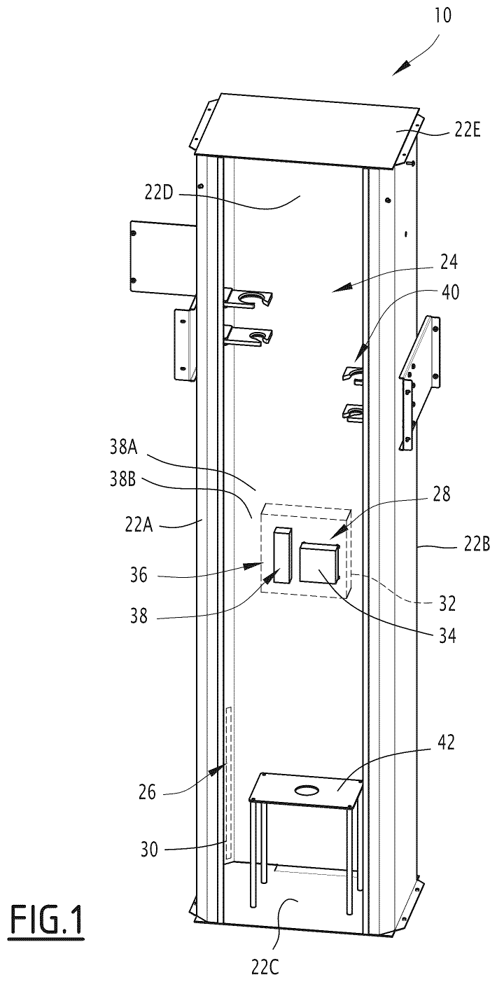

FIG. 1 is a schematic perspective view of an example of a disinfection device according to the invention;

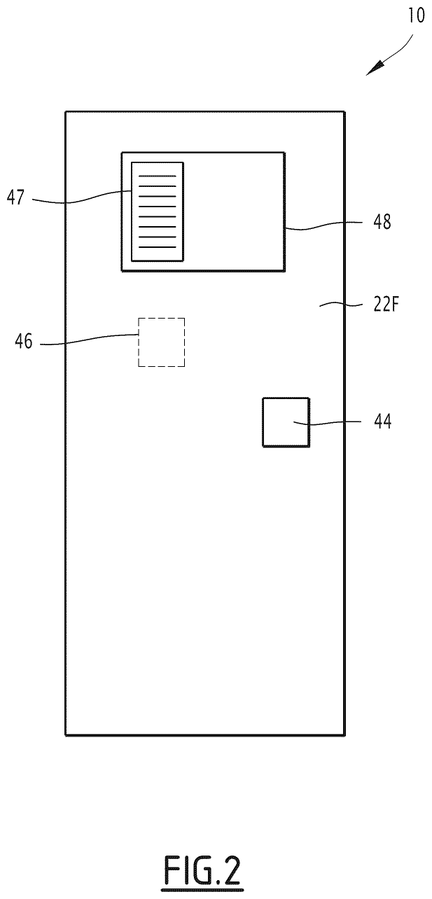

FIG. 2 is a schematic front view of an example of a disinfection device according to the invention;

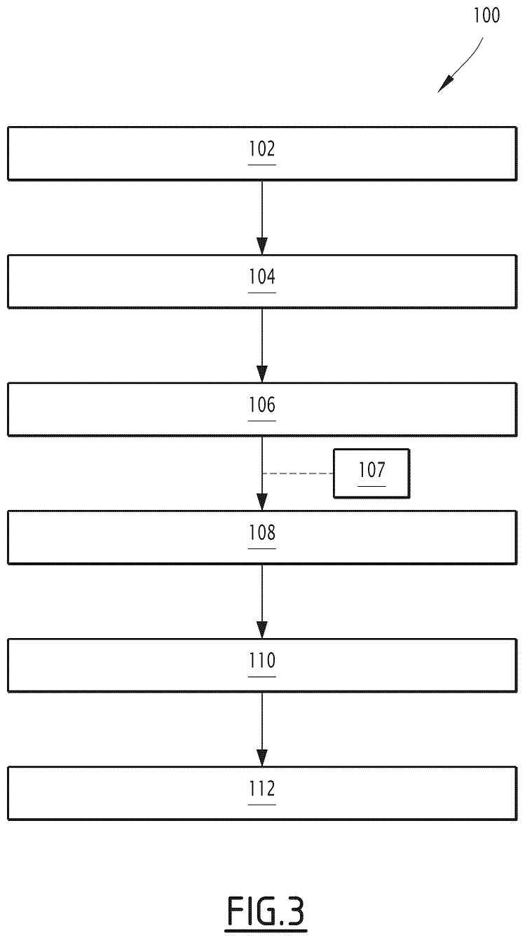

FIG. 3 is a flowchart of the steps of an example of a disinfection process according to the invention; and

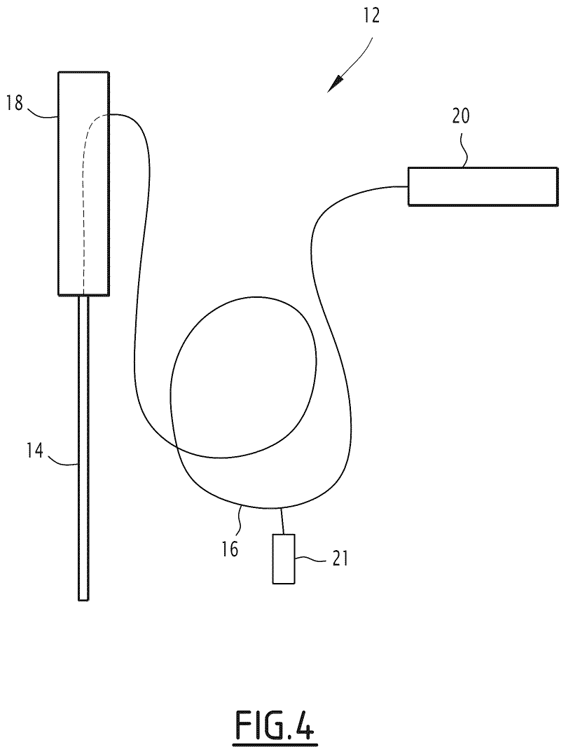

FIG. 4 is a schematic view of an example of a medical instrument suitable for being disinfected by the disinfection device from FIG. 1.

FIGS. 1 and 2 illustrate an example of a disinfection device 10 according to the invention. The disinfection device 10 is configured to disinfect all or part of a medical instrument 12.

FIG. 4 illustrates an example of a medical instrument 12 suitable for being disinfected by the disinfection device 10.

The medical instrument 12 comprises, for example, an active part 14. The active part 14 is, for example, a tube intended to be inserted into the patient, for example into a nostril of the patient.

Preferably, the medical instrument 12 comprises a handle 18 connected to the active part 14. The handle 18 is advantageously attached to one end of the active part 14 and is intended to be grasped by a user to facilitate the insertion of the active part 14 into the patient.

The medical instrument 12 also comprises a connecting part 16 and preferably also a connector 20.

The connecting part 16 is, for example, a connecting cable connecting the active part 14 to the connector 20.

The connector 20 is, for example, configured to be connected to a measurement and/or display unit, in particular to collect and/or display information captured by the active part 14.

The medical instrument 12 is, for example, a medical probe, in particular an ultrasound medical probe, and more particularly a transesophageal ultrasound (TEE) medical probe, or an endoscopic medical probe, for example of the “otorhinolaryngology” (ENT) type. The medical probe is, for example, a pediatric or adult probe.

Advantageously, an identification module 21 is attached to the medical instrument 12.

For example, as illustrated in FIG. 4, the identification module 21 is attached to the connecting part 16 of the medical instrument 12.

The identification module 21 is, for example, a tag.

The identification module 21 preferably comprises identification information of the medical instrument 12.

In particular, the identification module 21 comprises, for example, a radio-identification chip, also called an RFID (radio frequency identification) chip, containing this identification information.

The RFID chip is, for example, a low-frequency chip, a high-frequency chip, for example of the NFC (Near-Field Communication) type, or a UHF (Ultra High Frequency) chip.

Alternatively, the identification module 21 comprises a barcode.

This identification information comprises, for example, an identification number of the medical instrument 12 and/or the model of the medical instrument 12.

The disinfection device 10 comprises a disinfection volume 24 delimited by at least one wall, a UV radiation generation device 26, and an identification unit 28.

In a preferred embodiment, the disinfection device 10 comprises a plurality of walls 22A, 22B, 22C, 22D, 22E defining between them the disinfection volume 24.

More particularly, the walls 22A, 22B, 22C, 22D, 22E, 22F define between them a closed disinfection volume 24, for example of parallelepiped shape.

In the embodiment of the invention illustrated in FIGS. 1 and 2, the plurality of walls comprises, for example, a first lateral wall 22A and a second lateral wall 22B opposite each other, a bottom 22C, and a cover 22E opposite the bottom 22C, and also a base 22D defining a third lateral wall and a door 22F (not shown in FIG. 1) defining a fourth lateral wall facing the base 22D.

In particular, the cover 22E and the bottom 22C are, for example, perpendicular to the two lateral walls 22A, 22B. Similarly, the base 22D and the door 22F are, for example, perpendicular to the two lateral walls 22A, 22B, to the cover 22E, and to the bottom 22C.

In a non-illustrated variant, the disinfection device 10 comprises a single wall 22 delimiting the disinfection volume 24, which is, for example, cylindrical or spherical.

The UV radiation generation device 26, preferably UV-C, is configured to generate UV radiation in the disinfection volume 24.

For example, the UV radiation generation device 26 comprises a plurality of UV radiation sources 30 arranged inside the disinfection device 10, and for example on at least one of the walls 22, for example at least one of the lateral edges of the base 22D as illustrated in FIG. 1.

Each UV-C radiation source 30 is, for example, a mercury lamp generating UV-C radiation. In particular, each mercury lamp 30 is capable of using mercury to excite a gas and produce UV-C ultraviolet radiation.

By UV-C radiation, we mean radiation characterized by a wavelength between 200 and 280 nm.

Alternatively, each UV-C radiation source 30 is an LED (Light-Emitting Diode) lamp.

The identification unit 28 is positioned to identify the medical instrument 12 only when the medical instrument 12 is positioned in the disinfection volume 24.

By “identify,” we mean more particularly the reading of identification information of the medical instrument 12.

In particular, when the medical instrument 12 is positioned outside the disinfection volume 24, the identification unit 28 cannot identify it.

In the particular embodiment illustrated in FIGS. 1 and 2, the identification unit 28 is positioned in the disinfection volume 24.

More particularly, in the embodiment illustrated in FIG. 1, the identification unit 28 is attached to the base 22D of the disinfection device 10.

Alternatively, the identification unit 28 is positioned outside the disinfection volume 24. For example, the identification unit 28 is attached to an outer wall of the disinfection device 10 or is positioned at a distance from the disinfection device 10, for example by a support arm.

The identification unit 28 defines, for example, an identification volume 32.

By identification volume, we mean the volume wherein the identification unit 28 is capable of identifying an object positioned in said identification volume 32.

In particular, an object positioned outside the identification volume 32 cannot be identified by the identification unit 28.

The identification volume 32 is preferably at least partially comprised in the disinfection volume 24.

For example, the identification unit 28 comprises a radio-identification reader 34 arranged inside the disinfection volume 24. The radio-identification reader 34 is configured to read the identification information of the identification tag 21 attached to the medical instrument 12.

The radio-identification reader 34 preferably defines the identification volume 32.

The identification volume 32 extends, for example, around the radio-identification reader 34.

More precisely, the identification unit 28 is configured to identify the medical instrument 12 when the identification module 21 attached to the medical instrument 12 is positioned in the identification volume 32.

In particular, in the case of an identification module 21 with a low-frequency RFID chip intended to be read by the radio-identification reader 34, the identification volume 32 is, for example, about 10 cm³.

In contrast, in the case of an identification tag 21 with a UHF chip intended to be read by the radio-identification reader 34, by being “nearby,” the identification volume 32 is, for example, greater than 1 m³.

Advantageously, the disinfection device 10 also comprises a support 36 configured to hold the medical instrument 12 inside the disinfection volume 24.

Preferably, the support 36 is configured to hold the entire medical instrument 12 inside the disinfection volume 24.

In other words, when the medical instrument 12 is held by the support 36, all the elements constituting the medical instrument 12 are arranged inside the disinfection volume 24.

In particular, the active part 14, the connecting part 16, the handle 18, and the connector 20 are entirely held inside the disinfection volume 24.

More particularly, both ends of the connecting part 16 are inside the disinfection volume 24 when the medical instrument 12 is held by the support 36.

Advantageously, the support 36 is configured to position the identification module 21 attached to the medical instrument 12 inside the identification volume 32 of the identification unit 28.

To this end, as illustrated in FIG. 1, the support 36 comprises, for example, a first element 38 configured to position the identification module 21 attached to the medical instrument 12 inside the identification volume 32 of the identification unit 28.

More particularly, the first element 38 is configured to receive a portion of the connecting part 16, so as to position the identification module 21 attached to the connecting part 16 inside the identification volume 32.

For example, the first element 38 is attached to the base 22D of the disinfection device 10.

Advantageously, the first element 38 is configured to hold a portion of the connecting part 16 of the medical instrument 12, preferably the portion of the connecting part 16 carrying the identification module 21 so as to position the identification module 21 facing the identification surface 32.

In the particular embodiment illustrated in FIGS. 1 and 2, the support 36 comprises a plurality of secondary elements 40, 42. Each secondary element 40, 42 is preferably configured to hold a portion of the medical instrument 12, such as the active part 14, the handle 18, and/or the connector 20.

Advantageously, the disinfection device 10 also comprises an additional identification unit 44 for the user and/or the patient for acquiring identification information of the user of the medical instrument 12 and/or identification of the patient for whom the medical instrument 12 was used.

The additional identification unit 44 is preferably arranged outside the disinfection volume 24. In other words, the additional identification unit 44 is not inside the disinfection device 10.

For example, as illustrated in FIG. 2, the additional identification unit 44 is arranged on the external face of the door 22F of the disinfection device 10.

In a particular embodiment, the additional identification unit 44 is a barcode reader, for example configured to read a barcode of a badge of a user using the medical instrument 12 and/or the barcode of a file of a patient and/or the barcode of a file of a patient for which the medical instrument 12 was previously used.

The identification information of the user of the medical instrument 12 and/or identification of the patient comprises, for example, the surname and first name of the user, or of the patient.

Advantageously, the disinfection device 10 also comprises a processor 46 capable of implementing a disinfection cycle inside the disinfection volume.

The processor 46 is an electronic circuit designed to manipulate and/or transform data represented by electronic or physical quantities in the registers and/or memories of the processor into other similar data corresponding to physical data in the memories of registers or other types of display devices, transmission devices, or storage devices.

As specific examples, the processor 46 is implemented as a programmable logic component, such as an FPGA (Field Programmable Gate Array), or as an integrated circuit, such as an ASIC (Application Specific Integrated Circuit).

Alternatively, when the process is implemented as one or more software programs, i.e., as a computer program or computer program product, it is also capable of being recorded on a computer-readable medium (not shown). The computer-readable medium is, for example, a medium capable of storing electronic instructions and being coupled to a bus of a computer system. For example, the readable medium is an optical disk, a magneto-optical disk, a ROM memory, a RAM memory, any type of non-volatile memory (for example, FLASH or NVRAM), or a magnetic card. A computer program comprising software instructions is then stored on the readable medium.

Preferably, the processor 46 is configured to associate the identification information of the medical instrument 12 with disinfection characterization information.

The disinfection characterization information comprises, for example, the disinfection status.

In particular, the disinfection status comprises a timestamp of the disinfection and/or the state of the disinfection, for example, an “OK” or a “Success” if the disinfection cycle went well and a “NOK” or a “Failure” and the reason for the failure if the disinfection cycle failed.

To this end, the processor 46 is preferably connected to the identification unit 28, for example by a wired connection.

Preferably, the processor 46 is also configured not to trigger the disinfection cycle if no medical instrument 12 has been previously identified in the disinfection device 10 by the identification unit 28.

Furthermore, advantageously, the processor 46 is also configured to associate the identification information of the medical instrument 12 with the identification information of the user of the medical instrument 12 and/or identification of the patient for whom the medical instrument 12 was used.

To this end, the processor 46 is preferably connected to the additional identification unit 44 of the user and/or the patient, for example by a wired connection.

Advantageously, the identification information of the medical instrument 12, and preferably the disinfection characterization information and advantageously the identification information of the user of the medical instrument 12 and/or identification of the associated patient, are stored in a memory of the processor 46.

The processor 46 is preferably configured to generate a report 47 comprising all the associated information.

In particular, the report 47 comprises at least the identification information of the medical instrument 12 and the disinfection characterization information, and preferably also the identification information of the user of the medical instrument 12 and/or identification of the patient for whom the medical instrument 12 was used.

Advantageously, the disinfection unit 10 also comprises a display and/or printing and/or export unit 48 for the identification information of the medical instrument 12.

More particularly, the display and/or printing and/or export unit 48 is configured to display and/or print the report 47 generated by the processor 46 or to transmit the report 47 generated by the processor 46, for example, by digital export.

A disinfection process 100 for the medical instrument 12 in the disinfection device 10 will now be described with reference to FIG. 3.

Firstly, in a first step 102, a disinfection device 10 as described above is provided. The door 22F is open to give access to the disinfection volume 24.

Then, in a second step 104, a medical instrument 12 intended to be disinfected is positioned in the disinfection volume 24 of the disinfection device 10.

In particular, the medical instrument 12 is positioned entirely inside the disinfection volume 24 and is positioned to be held by the support 36.

More precisely, in a preferred embodiment of the invention, by means of the support 36, the identification module 21 attached to the medical instrument 12 is positioned in the identification volume 32 of the identification unit 28.

In a third step 106, the medical instrument 12 is identified in the disinfection volume 24 by the identification unit 28.

In particular, the identification unit 28, and more particularly the radio-identification reader 34, reads the identification information of the medical instrument 12, more particularly contained in the identification module 21.

During this third step 106, the identification information of the medical instrument 12 is preferably sent to the processor 46.

The door 22F is then closed.

In a particular embodiment, the process 100 also comprises a step 107 of acquiring identification information of the user and/or the patient, in particular by the additional identification unit 44.

In a step 108, the disinfection cycle of the medical instrument 12 is launched only when identification information has been read by the identification unit 28.

In particular, the processor 46 launches the disinfection cycle only if it has received identification information of the medical instrument 12.

The disinfection cycle is adapted to disinfect all or part of the medical instrument 12 positioned in the disinfection volume 24.

More precisely, the processor 46, for example, gives a command order to the UV radiation generation device 26. The command order comprises, for example, the duration of the cycle and the dose of UV-C radiation.

Advantageously, the processor 46 adapts its command order to the UV radiation generation device 26 based on the identified medical instrument 12.

Advantageously, the process 100 comprises a step 110 of associating the identification information of the medical instrument 12 with the disinfection characterization information, and preferably also with the identification information of the user and/or the patient collected in step 108.

Preferably, the identification information of the medical instrument 12, and advantageously also the information during this step 110, are stored.

During this step 110, the processor 46, for example, generates the report 47 comprising all the associated information.

Preferably, the process 100 also comprises a step 112 of printing and/or displaying and/or exporting these associated information. For example, this step 112 comprises printing a physical and/or digital ticket comprising the report 47. By reference, this step 112 also comprises saving this information.

Such a disinfection device 10 is particularly advantageous for the disinfection of medical instruments 12, and in particular medical probes.

In particular, by means of the identification unit 28 positioned in the disinfection device 10, the medical instrument 12 is identified only when it is positioned in the device. Thus, the identified instrument 12 is certainly the same as the one being disinfected.

Furthermore, the association of the identification information of the medical instrument 12 with other information, notably those of disinfection characterization and possibly those of identification of the user and/or the patient, ensures improved traceability of the use and disinfection of the medical instrument 12.

In addition, the generation of a report 47 containing all this information provides precise and reliable documentation of the use and disinfection of the medical instrument 12, which facilitates the traceability of the use and disinfection of the medical instrument 12.

The person skilled in the art will understand that the previously described embodiments and variants can be combined with each other as long as they are technically compatible.

1. A disinfection device for a medical instrument comprising:
a disinfection volume delimited by at least one wall,
a UV radiation generation device configured to generate UV radiation in the disinfection volume, and
an identification unit positioned to read identification information of a medical instrument only when the medical instrument is positioned in the disinfection volume.

2. The disinfection device according to claim 1, comprising a support configured to hold the medical instrument inside the disinfection volume.

3. The disinfection device according to claim 2, wherein the medical instrument comprises an identification module comprising the identification information of the medical instrument, the identification unit defining an identification volume, the support being configured to hold the medical instrument so as to position the identification module attached to the medical instrument in the identification volume of the identification unit.

4. The disinfection device according to claim 3, wherein the identification unit of the medical instrument comprises a radio-identification reader defining the identification volume.

5. The disinfection device according to claim 1, comprising a processor configured to associate the identification information of the medical instrument with disinfection characterization information.

6. The disinfection device according to claim 5, wherein the disinfection characterization information comprises at least the disinfection status.

7. The disinfection device according to claim 1, comprising an additional identification unit for the user and/or the patient arranged outside the disinfection volume for acquiring identification information of the user of the medical instrument and/or identification of the patient for whom the medical instrument was used.

8. The disinfection device according to claim 1, also comprising a display and/or printing and/or export unit for the identification information of the medical instrument.

9. The disinfection process for a medical instrument comprising the following successive steps:
a. providing a device according to claim 1,
b. positioning the medical instrument inside the disinfection volume,
c. reading the identification information of the medical instrument in the disinfection volume by the identification unit, and
d. starting a disinfection cycle of the medical instrument only when identification information has been read by the identification unit.

10. The process according to claim 9, also comprising a step of associating the identification information of the medical instrument with disinfection characterization information.
